(19) United States
(12) Patent Application Publication
(10) Pub. No.: US 2014/0341880 A1
(43) Pub. Date: Nov. 20, 2014

(54) RISK PREDICTORS FOR ADVERSE PERINATAL OUTCOMES

(71) Applicant: Nationwide Children's Hospital, Inc., Columbus, OH (US)

(72) Inventors: Sheryl Sue Justice, Westerville, OH (US); Santiago Partida-Sanchez, Galloway, OH (US)

(21) Appl. No.: 14/345,132

(22) PCT Filed: Sep. 17, 2012
(86) PCT No.: PCT/US12/55749

§ 371 Date: Mar. 14, 2014

Related U.S. Application Data
(60) Provisional application No. 61/535,056, filed on Sep. 15, 2011.

(51) Int. Cl. G01N 33/68

(52) U.S. Cl. CPC G01N 33/689; G01N 2333/525; G01N 2333/54; G01N 2333/5412
USPC 424/94.64; 436/501; 424/94.65; 514/11.5

(57) ABSTRACT

Methods for assessing a risk of experiencing an adverse perinatal outcome in a subject are described. The methods include determining a level of at least one cytokine in a biological sample from the subject; and comparing the level of the at least one cytokine in the subject's biological sample with a predetermined value based on levels of the at least one cytokine in a biological sample from a population of control subjects, wherein a subject whose level of at least one cytokine is greater than the predetermined line value is at risk of experiencing an adverse perinatal outcome.

### CROSS-REFERENCE TO RELATED APPLICATIONS

This application claims priority to International Application No. PCT/US2012/055749, filed Sep. 17, 2012, which in turn claims the benefit of U.S. Provisional Patent Application No. 61/535,056, filed Sep. 15, 2011, which is incorporated herein by reference in its entirety.

### GOVERNMENT FUNDING

The present invention was made with government support under National Institutes of Health Grant No. 1R01AIO92117-01A1. The U.S. Government has certain rights in this invention.

### BACKGROUND

One in every eight births in the United States and one in every ten worldwide births is preterm. The American College of Obstetrics and Gynecology recommends screening of all pregnant women for urinary tract infections (UTIs) due to the increased frequencies of asymptomatic and acute bacteriuria, and an association with both UTIs and adverse perinatal outcomes. Given the high rate of both asymptomatic and symptomatic bacteriuria during pregnancy and lack of predictive diagnostic, the precise number of preterm deliveries due to UTI is unknown. Current studies estimate that up to 25% of all preterm deliveries result from maternal UTI. Infants born prematurely due to maternal UTI demonstrate increased morbidity and mortality within the first year of life, and are at greater risk for lifelong learning disabilities and chronic illnesses. To date there are no known diagnostic tools that will indicate the risk for adverse perinatal outcomes following a maternal infection. Andrews et al., Am. J. Perinatol. 17, 357 (2000). There is a critical need to develop a test that will identify which pregnancies are at risk for adverse outcomes. The ability to identify a fetus at risk for an adverse perinatal outcome will allow prevention of adverse perinatal outcomes, therapeutic intervention to increase gestational length, and increase the overall health of infants.

### SUMMARY

Disclosed herein are methods for assessing a risk of experiencing an adverse perinatal outcome in a subject comprising determining a level of at least one cytokine in a biological sample from the subject by analyzing the biological sample using a cytokine analytic method, and comparing the level of the at least one cytokine in the subject's biological sample with a predetermined value based on levels of the at least one cytokine in a biological sample from a population of control subjects, wherein a subject whose level of at least one cytokine is greater than the predetermined value is at increased risk of experiencing an adverse perinatal outcome. In some exemplary embodiments, the at least one cytokine is a pro-inflammatory cytokine. In some exemplary embodiments, the pro-inflammatory cytokine is selected from the group consisting of interleukin-6, interleukin-17, and tumor necrosis factor alpha (TNF-α). In some exemplary embodiments, the at least one cytokine is a circulatory cytokine, urinary cytokine, or both.

In some exemplary embodiments, the biological sample is selected from the group consisting of blood, serum, and urine. In some exemplary embodiments, the subject has been diagnosed with a urinary tract infection. In some exemplary embodiments, the population of control subjects is pregnant females. In some exemplary embodiments, the pregnant females are apparently healthy pregnant females. In some exemplary embodiments, the apparently healthy pregnant females have been diagnosed as not having an infection. In some exemplary embodiments, the diagnosis can be made by employing a urinalysis at the end of the first trimester. In some exemplary embodiments, the infection is a urinary tract infection. In some exemplary embodiments, the control or baseline value is a single normalized value or a range of normalized values and is based on the at least one cytokine level in comparable biological samples from the control subjects. In other exemplary embodiments, the control or baseline value is a single representative value or a range of representative values and is based on the at least one cytokine level in comparable biological samples from the control subjects.

In some exemplary embodiments, the methods may further comprise administering an effective amount of a therapeutic agent to reduce the level of at least one cytokine. In some exemplary embodiments, the therapeutic agent is selected from the group consisting of cathepsin Q, prolactin, leptin receptor, cathepsin 3, cathepsin 6, cathepsin M, cathepsin J, cathepsin R, ceacam, and anti-inflammatory agents. In some exemplary embodiments, the anti-inflammatory agents are selected from NSAIDS and IL-10.

Also disclosed herein are methods for evaluating a therapy in a subject suspected of being or diagnosed as being at risk for an adverse perinatal outcome that includes determining a level of at least one cytokine in a biological sample obtained from the subject using a cytokine analytic method prior to therapy to decrease the risk of an adverse perinatal outcome and during or after therapy, comparing the level of at least one cytokine in a biological sample obtained from the subject prior to therapy to the level of at least one cytokine in a biological sample obtained from the subject during or after therapy, wherein a decrease in the at least one cytokine level in the subject's biological sample taken after or during therapy as compared to the at least one cytokine level in the subject's biological sample taken before therapy is indicative of a positive effect of the therapy.

Also contemplated herein are kits comprising one or more reagents for determining a level of at least one cytokine in a subject, and information for assessing the subject's risk of having an adverse perinatal outcome.

Additional features and advantages of the exemplary embodiments will be set forth in part in the description that follows, and in part will be obvious from the description, or may be learned by practice of the exemplary embodiments disclosed herein. The objects and advantages of the exemplary embodiments disclosed herein will be realized and attained by means of the elements and combinations particularly pointed out in the appended claims. It is to be understood that both the foregoing brief summary and the following detailed description are exemplary and explanatory only and are not restrictive of the embodiments disclosed herein or as claimed.

### BRIEF DESCRIPTION OF THE FIGURES

The accompanying drawings, which are incorporated in and constitute a part of this specification, illustrate some embodiments disclosed herein, and together with the description, serve to explain principles of the exemplary embodiments disclosed herein.

FIG. 1 provides a graph showing perinatal outcomes in the presence and absence of maternal UTI. (A) Weight (grams) of each individual offspring from pregnant mice that received sham treatment (white) or experimental UTI (gray) at 48 h (10 mice each cohort), 96 h (10 mice each cohort), and delivery (8 mice each cohort). Box portion of the plot represents 95% of the samples with the range of samples indicated by the external bars; the horizontal bar within the box depicts the median and the mean values are indicated in the text. (B) The number of pups delivered for each mother is depicted. Statistical significance was determined using a two-tailed Mann-Whitney test for internal comparisons of each time point indicated in each panel.

FIG. 2 provides a graph showing PMN and macrophage infiltration in uteroplacental tissues. The magnitude of the particular cell type of interest is reported as a percentage of live leukocytes within each individual organ indicated from pregnant mice that received sham infection (white) or experimental UTI (gray) at 48 h (10 mice each cohort), 96 h (10 mice each cohort), and delivery (8 mice each cohort). Abbreviations: PMN, polymorphonuclear neutrophil; Mac, macrophage. Bars indicate standard deviation. Statistical significance determined using a two-tailed Mann-Whitney for internal comparisons within each time point indicated in each panel

FIG. 3 provides a graph showing the presence of dendritic cells in uteroplacental tissues. The magnitude of the particular cell type of interest is reported as a percentage of live leukocytes within each individual organ indicated from pregnant mice that received sham infection (white) or experimental UTI (gray) at 48 h (10 mice each cohort), 96 h (10 mice each cohort), and delivery (8 mice each cohort. Statistical significance determined using a two-tailed Mann Whitney for internal comparisons within each time point, no significance observed. Abbreviations: mDC, mature dentritic cell; iDC, immature dendritic cell.

FIG. 4 provides a graph showing serum cytokine profiles during pregnancy and UTI. The magnitude of serum cytokines (pg/ml) of cytokines at 96 h (A) and delivery (B) of mothers that received sham infection (white) or experimental UTI (gray). The serum taken from each individual mouse is measured separately. The number of mice used in each cohort at 96 h and delivery were 10 and 8, respectively. Statistical significance was determined using two-tailed Mann-Whitney (**, p<0.04; ***, p<0.008; ****, p=0.0003).

### DETAILED DESCRIPTION

The exemplary embodiments disclosed herein will now be described by reference to some more detailed exemplary embodiments, with occasional reference to the accompanying drawings. These exemplary embodiments may, however, be embodied in different forms and should not be construed as limited to the embodiments set forth herein. Rather, these exemplary embodiments are provided so that this disclosure will be thorough and complete, and will fully convey the scope of the exemplary embodiments to those skilled in the art.

Unless otherwise defined, all technical and scientific terms used herein have the same meaning as commonly understood by one of ordinary skill in the art to which these exemplary embodiments belong. The terminology used in the description herein is for describing particular exemplary embodiments only and is not intended to be limiting of the exemplary embodiments. As used in the specification and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present embodiments. At the very least, and not as an attempt to limit the application of the doctrine of equivalents to the scope of the claims, each numerical parameter should be construed in light of the number of significant digits and ordinary rounding approaches.

As used herein, the term “diagnosis” can encompass determining the nature of disease or condition in a subject, as well as determining the severity and probable outcome of disease or episode of disease and/or prospect of recovery (prognosis). “Diagnosis” can also encompass diagnosis in the context of rational therapy, in which the diagnosis guides therapy, including initial selection of therapy, modification of therapy (e.g., adjustment of dose and/or dosage regimen), and the like.

The terms “individual,” “host,” “subject,” and “patient” are used interchangeably herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. Due to the interest in predicting the likelihood of an adverse perinatal outcome, subjects are pregnant females of these species, and in particular pregnant human females

A method for assessing a risk of experiencing an adverse perinatal outcome in a subject comprising: (a) determining a level of at least one cytokine in a biological sample from the subject by analyzing the biological sample using a cytokine analytic method; and (b) comparing the determined level of the at least one cytokine in the subject's biological sample with at least one predetermined value, wherein a subject whose level of at least one cytokine is greater than the predetermined value is at increased risk of experiencing an adverse perinatal outcome.

An adverse perinatal outcome, as defined herein, refers to a preterm delivery, miscarriage, fetal death, fetal injury, or low birth weight. Pregnancy is considered “at term” when gestation has lasted 37 complete weeks (occurring at the transition from the 37th to the 38th week of gestation) for human female subjects, but is less than 42 weeks of gestational age. Delivery before completion of 37 weeks (259 days) is considered preterm. The gestational period for other mammals is known to those skilled in the art. Preterm delivery includes delivery up to one week before term, up to two weeks before term, up to three weeks before term, up to one month before term, up to two months before term, and two months or more before term.

Cytokines are small cell-signaling protein molecules that are secreted by numerous cells and are a category of signaling molecules used extensively in intercellular communication. Cytokines can be classified as proteins, peptides, or glycoproteins. Each cytokine has a matching cell-surface receptor. Subsequent cascades of intracellular signalling result from association of the cytokine with its matching receptor, thereby altering cell function. This may include the upregulation and/or downregulation of several genes and their transcription factors. Cytokines have been classed as lymphokines, interleukins, and chemokines, based on their function, cell of secretion, and/or target of action.

The methods described herein include determining the level of at least one cytokine. For example, the method can include determining the level of a single cytokine, or it can include a determination of the level of two or more cytokines. In some embodiments, the cytokine is a circulatory cytokine, a urinary cytokine, or both. In other embodiments, the cytokine is a pro-inflammatory cytokine. In further embodiments, the pro-inflammatory cytokine is selected from the group consisting of interleukin-6, interleukin-17, and tumor necrosis factor alpha (TNF-α).

Another aspect of the invention provides a method for evaluating therapy in a subject suspected of being or diagnosed as being at increased risk for an adverse perinatal outcome. This method includes (a) determining a level of at least one cytokine in a biological sample obtained from the subject prior to therapy to decrease the risk of an adverse perinatal event and during or after therapy using a protein analytic method, and (b) comparing the level of at least one cytokine in a biological sample obtained from the subject prior to therapy to the level of at least one cytokine in a biological sample obtained from the subject during or after therapy, wherein a decrease in the at least one cytokine level in the subject's biological sample taken after or during therapy as compared to the at least one cytokine level in the subject's biological sample taken before therapy is indicative of a positive effect of the therapy.

### Biological Samples

Biological samples include, but are not necessarily limited to biological fluids such as urine and blood-related samples (e.g., whole blood, serum, plasma, and other blood-derived samples), urine, cerebral spinal fluid, bronchoalveolar lavage, and the like. A preferred biological sample for assessing the risk of experiencing an adverse perinatal outcome is urine. Another example of a biological sample is a tissue sample. The levels of the one or more cytokines can be assessed either quantitatively or qualitatively, usually quantitatively. The levels of cytokines can be determined either in vivo or in vitro.

A biological sample may be fresh or stored (e.g. blood or blood fraction stored in a blood bank). Samples can be stored for varying amounts of time, such as being stored for an hour, a day, a week, a month, or more than a month. The biological sample may be a biological fluid expressly obtained for the assays of this invention or a biological fluid obtained for another purpose which can be subsampled for the assays of this invention.

### Methods for Measuring Levels of Cytokines

The presence of one or more cytokines may be determined by any of a variety of standard cytokine analytic methods (i.e., methods suitable for determining cytokine levels) known in the art. As noted herein, cytokines are proteins, peptides, or glycoproteins, and therefore their levels can be determined by methods suitable for evaluating levels of these types of compounds. These methods include immunoassays such as enzyme-linked immunosorbent assays (ELISA), mass spectrometry, and other techniques known to one of ordinary skill in the art. The devices to carry out cytokine analysis can be collectively referred to herein as cytokine detectors. Results from an immunoassay can further be evaluated using flow cytometry for a rapid determination of cytokine levels.

The level of the one or more cytokines in a subject can be measured using an analytic device, which is a machine including a detector capable of identifying cytokines and substantial fragments thereof. The analytic device may be a spectrometric device, such as a mass spectrometer, an ultraviolet spectrometer, or a nuclear magnetic resonance spectrometer. A spectrometer is a device that uses a spectroscopic technique to assess the concentration or amount of a given species in a medium such as a biological sample (e.g., a biological fluid). The analytic device used to measure the levels of cytokines can be either a portable or a stationary device. In addition to including equipment used for detecting cytokines, the analytic device can also include additional equipment to provide purification (i.e., physical separation) of analytes prior to analysis. For example, if the analyte detector is a mass spectrometer, it may also include a high performance liquid chromatograph (HPLC) or gas chromatograph (GC) to purify the cytokines before their detection by mass spectrometry, or it may be preferable to purify the protein using gel electrophoresis.

As indicated herein, mass spectrometry-based methods can be used to assess levels of one or more cytokines in a biological sample. Mass spectrometers include an ionizing source (e.g., electrospray ionization), an analyzer to separate the ions formed in the ionization source according to their mass-to-charge (m/z) ratios, and a detector for the charged ions. In tandem mass spectrometry, two or more analyzers are included. Such methods are standard in the art and include, for example, HPLC with on-line electrospray ionization (ESI) and tandem mass spectrometry.

Once the levels of the one or more cytokines have been determined, they can be displayed in a variety of ways. For example, the levels of cytokines can be displayed graphically on a display as numeric values or proportional bars (i.e., a bar graph) or any other display method known to those skilled in the art. The graphic display can provide a visual representation of the amount of one or more cytokines in the biological sample being evaluated. In some embodiments, the levels of one or more cytokines in the biological sample obtained from a subject is compared to a predetermined value. In addition, in some embodiments, the analytic device can also be configured to display a comparison of the levels of one or more cytokines in the subject's biological sample to a predetermined value based on levels of cytokines in comparable biological sample from a reference cohort.

### Predetermined Values

In one embodiment, the predetermined value is related to the value used to characterize the levels of cytokines thereof in the biological sample obtained from a subject. Thus, if the levels of cytokines are an absolute value such as the units of cytokines per milliliter of urine, the predetermined value is also based upon the units of cytokines per ml of urine in individuals in the general population or a select population of subjects. Similarly, if the levels of cytokines, and the combination thereof are a representative value such as an arbitrary unit obtained from a cytogram, the predetermined value is also based on the representative value.

The predetermined value may take a variety of forms. In some embodiments, the predetermined value is a single normalized value or a range of normalized values and is based on the at least one cytokine level in comparable biological samples from the control subjects. In other embodiments, the predetermined value is a single representative value or a range of representative values and is based on the at least one cytokine level in comparable biological samples from the control subjects. The predetermined value may also be a single cut-off value, such as a median or mean. In some embodiments, the predetermined value may be established based upon comparative groups such as where risk in one defined group is double the risk of another defined group. In another embodiment, the predetermined value may be a range, for example, where the general population is divided equally (or unequally) into groups, such as a low risk group, a medium risk group and a high-risk group, or into quadrants, the lowest quadrant being individuals with the lowest risk the highest quadrant being individuals with the highest risk.

The predetermined value may be derived by determining the level of cytokines in the general population. Alternatively, the predetermined value can be derived by determining the level of cytokines in a select population, such as pregnant females or apparently healthy pregnant female humans. For example, an apparently healthy pregnant female human population may have a different normal level of cytokines than will a population of healthy non-pregnant female humans or a population of pregnant female humans with a urinary tract infection. Accordingly, the predetermined values selected may take into account the category in which an individual falls. Appropriate ranges and categories may be selected with no more than routine experimentation by those of ordinary skill in the art.

In some embodiments, the select population may be comprised of apparently healthy subjects. “Apparently healthy,” as used herein, means individuals who have not previously had any signs or symptoms indicating the presence of other risk factors associated with an adverse perinatal outcome. Examples of risk factors associated with an adverse perinatal outcome include having a previous premature birth, pregnancy with twins, triplets or other multiples, an interval of less than six months between pregnancies, conceiving through in vitro fertilization, problems with the uterus, cervix or placenta, smoking cigarettes, drinking alcohol or using illicit drugs, poor nutrition, infections, particularly of the amniotic fluid and lower genital tract, high blood pressure, diabetes, being underweight or overweight before pregnancy, stressful life events, such as the death of a loved one or domestic violence, multiple miscarriages or abortions, physical injury or trauma, or an unusually shaped uterus. In a further embodiment, an apparently healthy subject is one who does not have a urinary tract infection.

In some embodiments, predetermined values for the levels of cytokines, for example mean levels, median levels, or “cut-off” levels, are established by assaying a large sample of individuals in the general population or a select population and using a statistical model such as the predictive value method for selecting a positivity criterion or receiver operator characteristic curve that defines optimum specificity (highest true negative rate) and sensitivity (highest true positive rate). A “cutoff” value may be determined for the presence of cytokines.

Comparison of Cytokine Levels in the Biological Sample from the Subject to the Predetermined Value

The levels of each risk predictor (i.e., one or more cytokines) in a subject's biological sample may be compared to a single predetermined value or to a range of predetermined values. If the level of the present risk predictor in the subject's biological sample is greater than the predetermined value or range of predetermined values, the subject is at greater risk of having an adverse perinatal outcome than individuals with levels comparable to or below the predetermined value or predetermined range of values. In contrast, if the level of the present risk predictor in the subject's biological sample is below the predetermined value or range of predetermined range, the subject is at a lower risk of having an adverse perinatal outcome than individuals with levels comparable to or above the predetermined value or range of predetermined values.

The comparison can be conducted by any suitable method known to those skilled in the art. For example, the comparison can be carried out mathematically or qualitatively by an individual operating the analytic device or by another individual who has access to the data provided by the analytic device. Alternately, the steps of determining and comparing the levels of cytokines can be carried out electronically (e.g., by an electronic data processor).

For example, a subject who has a higher level of one or more cytokines as compared to the predetermined value is at high risk of developing an adverse perinatal outcome, and a subject who has a lower level of cytokines as compared to the predetermined value is at low risk of developing an adverse perinatal outcome. The extent of the difference between the subject's risk predictor levels and predetermined value is also useful for characterizing the extent of the risk and thereby determining which subject's would most greatly benefit from certain aggressive therapies. In those cases, wherein the predetermined value ranges are divided into a plurality of groups, such as the predetermined value ranges for individuals at high risk, average risk, and low risk, the comparison involves determining into which group a subject's level of the relevant risk predictor falls.

The method can also include the step of providing a report indicating the subject is in need of therapy if levels of cytokines are higher than at least one of the one or more predetermined values. For example, the apparatus for carrying out the method can include a processor coupled to the protein detector and adapted to quantify the data representing the signals from the detector, and adapted to perform the multivariate statistical analysis, compare the output value to the first reference value and the second reference value, and calculate the risk score; and an output display coupled to the processor and configured to report the risk score.

The present diagnostic tests may be useful for determining if and when therapeutic agents for avoiding an adverse perinatal outcome should or should not be prescribed for a subject. For example, the method can further include administering a therapeutically effective amount of an anti-inflammatory agent or progestational agent to a subject. Subjects with values of cytokines (μg/mL urine) above a certain cutoff value, or that are in the higher tertile or quartile of a “normal range,” could be identified as those in need of more aggressive intervention. Examples of therapeutic agents include progestational agents, cytokine antagonists, and anti-inflammatory agents. Preferred therapeutic agents include cathepsin Q, prolactin, leptin receptor, cathepsin 3, cathepsin 6, cathepsin M, cathepsin J, cathepsin R, ceacam, and anti-inflammatory agents.

In another embodiment, the present invention relates to kits that include reagents for assessing levels of one or more cytokines in biological samples obtained from a subject. Such assays have appropriate sensitivity with respect to predetermined values selected on the basis of the present diagnostic tests. In certain embodiments, the kits also include printed materials such as instructions for practicing the present methods, or information useful for assessing a subject's risk of developing an adverse perinatal outcome. Examples of such information include, but are not limited to cut-off values, sensitivities at particular cut-off values, as well as other printed material for characterizing risk based upon the outcome of the assay. In some embodiments, such kits may also comprise control reagents, e.g., known amounts of cytokines

### Evaluation of Therapeutic Agents

The present diagnostic tests are also useful for evaluating the effect of therapeutic agents on subjects who have been diagnosed as having an increased risk of experiencing an adverse perinatal outcome. An additional aspect of the invention provides a method for evaluating the effect of therapy in a subject diagnosed as having an increased risk of experiencing an adverse perinatal outcome. Examples of therapy include treatment with progestational agents, cytokine antagonists, or anti-inflammatory agents. In particular, the method is suitable for evaluating the effect of therapy on the likelihood of an adverse perinatal outcome.

The method includes determining levels of cytokines in a biological sample taken from the subject prior to therapy to decrease the risk of an adverse perinatal event and determining levels of cytokines in a corresponding biological sample taken from the subject during or following therapy, wherein a decrease in levels of cytokines in the sample taken after or during therapy as compared to levels of cytokines in the sample taken before therapy is indicative of a positive effect of the therapy on the likelihood of experiencing an adverse perinatal outcome in the treated subject.

In specific embodiments, the level of one or more cytokines in the biological sample can determined. Suitable biological samples include those described herein, such as urine. The levels of cytokines in the method of evaluating therapy can be determined using any suitable cytokine analytic method. Examples of cytokine analytic methods are described herein, and include immunoassay and cytokine separation following by cytokine analysis using mass spectrometry

Such evaluation comprises determining the levels of one or more of the present risk predictors including one or more cytokines in a biological sample taken from a subject prior to administration of the therapeutic agent and a corresponding biological fluid taken from a subject following administration of the therapeutic agent. A decrease in the level of the selected risk factor in the sample taken after administration of the therapeutic as compared to the level of the selected risk factor in the sample taken before administration of the therapeutic agent is indicative of a positive effect of the therapeutic agent on the risk the subject will experience an adverse perinatal outcome.

Progestational agents are suitable therapeutic agents, and include progesterone, calcium channel blockers, beta-agonists, cytokine antagonists, prostaglandin inhibitors, corticosteroids, and various additional agonists for preventing placental breakdown and/or improving placental function. For a discussion of various progestational agents, see Ables et al., J. Fam Pract, 54(3), p. 245-252 (2005), the disclosure of which is incorporated by reference herein. In further embodiments, the therapeutic agent is selected from the group consisting of cathepsin Q, prolactin, leptin receptor, cathepsin 3, cathepsin 6, cathepsin M, cathepsin J, cathepsin R, ceacam, and anti-inflammatory agents.

The following example is included for purposes of illustration and is not intended to limit the scope of the invention.

### EXAMPLE

### Example 1

### Intrauterine Growth Restriction is a Direct Consequence of Localized Maternal Uropathogenic Escherichia coli Cystitis

Pregnancy is a unique situation in which the fetus carries both maternal (“self”) and paternal (“non-self”) antigens. While the immune system normally functions to attack non-self stimuli, successful gestation requires the maternal immune response to “ignore” paternal antigens produced by the fetus (fetal tolerance). Maternal immune cells come into direct contact with fetal cells at the decidua, a highly specialized mucous membrane that contains regulatory immune effectors involved in fetal tolerance. While the mechanisms of tolerance between mother and fetus are not completely understood, it is clear that dendritic cells (DCs), T cells and natural killer (NK) cells play crucial roles at the materno-fetal interface and in maternal tissues to ensure that pregnancy proceeds successfully. Blois et al., Biol Reprod 77, 590 (October, 2007). Dendritic cells are only partially activated as the maternal immune system reacts to the implanted and developing fetus. These immature dendritic cells (iDCs) migrate to local lymph nodes and induce T cell differentiation into Th2/3 and regulatory T cells (Treg). Th2/3 cells produce cytokines (e.g. IL-10, IL-4,) promote a tolerant environment suitable for the developing fetus.

Maternal infections contribute to nearly 40% of all preterm deliveries. Previously published murine models have been useful to delineate the delicate balance of immunological changes that lead to adverse perinatal outcomes due to maternal intrauterine infection. Elovitz et al., Trends Endocrinol Metab 15, 479 (2004). While the maternal immune response goes to great lengths to protect the fetus from immunological attack, there are situations where the fetal tolerance is broken leading to adverse perinatal outcomes. Over a decade ago Matzinger postulated that immunological attack of non-self only occurs when the non-self poses potential damage to self (e.g. infection). While it is known that various cellular and soluble immune effectors contribute to fetal tolerance and thus, a successful pregnancy, these effectors may negatively affect the developing fetus when there is an inflammatory stimulus (e.g. infection). Specifically, in response to an inflammatory/infectious stimulus, interferon gamma (IFN-γ) and tumor necrosis factor (TNF) fully promote DC maturation during pregnancy. Thus, mature DCs (mDCs) may travel systemically and, in the presence of IL-12, induce T cell differentiation into Th1 type cells. Th1 cells then produce more IL-12, IFN-γ and TNF, which result in an inflammatory, unfavorable environment, for a developing fetus. Blois et al., Biol Reprod 77, 590 (2007). Certainly local intrauterine infections can elicit sufficient inflammatory cytokines that recruit inflammatory cells, which in turn make the intrauterine environment inhospitable to a developing fetus. P. Matzinger, Annual review of immunology 12, 991 (1994). However, it is not clear how in the presence of a localized extrauterine infection, such an acute cystitis, the detrimental signal (or chain of signals) may be transported to the materno-fetal interface causing low birth weight and/or preterm birth.

Urinary tract infections (UTIs) affect almost 50% of all women, manifesting in a variety of clinical presentations (i.e. asymptomatic bacteriuria, cystitis, and pyelonephritis). While many UTIs generally have a mild clinical course with few sequelae in the general population, even covert bacteriuria places the gestating female at risk for low birth weight offspring and preterm birth. Several clinical studies have detailed the adverse perinatal effects UTIs have on the developing fetus, with Mazor-Dray in 2009 concluding that UTIs act as an independent risk factor for preterm delivery and intrauterine growth restriction-low birth weight (IUGR-LBW). Gilstrap et al., Obstet. Gynecol. Clin. North Am. 28, 581 (2001). Maternal UTI raises the risk of prematurity and low birth weight. In fact, ACOG recommends screening all pregnant women for UTIs during the 1^{st} trimester (standard of care) and subsequent antenatal visits (suggested for high-risk patients). The clinical evidence indicates a critical need to elucidate the mechanisms that underlie the maternal UTI-mediated adverse outcomes to reduce the number of premature deliveries.

The use of murine models has re-defined UTIs caused by uropathogenic Escherichia coli (UPEC), the most common causative agent, as a complex intracellular infection. Growth of UPEC involves successive morphological and physiological changes during a complex intracellular developmental cycle that is critical for the establishment of lower urinary tract infections. An acute episode of cystitis culminates in the establishment of a latent or chronic infection. Evidence for each of the stages is present in human urine samples and human biopsies of patients colonized with UPEC, which validates the observations made in the murine model. Both human and murine studies have demonstrated that IL-8 plays a significant role in polymorphonuclear neutrophil (PMN) migration to the UPEC-infected urinary tract. Sivick et al., Infect Immun 78, 568 (2010). Data from UPEC-infected mice have also demonstrated increased bladder levels of IL-17, TNF-α, and IL-6, the latter of which has been corroborated in human cell lines and patient urine. Ingersoll et al., Cell Microbiol 10, 2568 (2008), Hedges et al., Infect Immun 59, 421 (1991).

Kaul et al. presented a murine model of pyelonephritis-induced preterm birth and low birth weight offspring while investigating the virulence of an adhesin possessed by E. coli. Kaul et al., Infect Immun 67, 5958 (1999). The authors used immunocompromised mice and demonstrated that systemic bacterial burden induced fetal sepsis and death. Yet disseminated or intrauteral infections are not required for adverse perinatal outcomes in humans, here, the inventors hypothesize that a cascade of immunological events must occur in response to a UTI that affects uninfected organs such as the uteroplacental tissue. To this end, a novel murine model was developed for UTI-mediated adverse perinatal outcomes (i.e. preterm birth, IUGR-LBW), and analyzed the cellular and/or soluble immune effectors that contribute to perinatal morbidity. Moreover, potential markers were identified that can be used to predict relative risk to the fetus following maternal UTI.

### Results

Maternal UTI Results in Low Birth Weight Offspring.

Prematurity is defined by two clinical outcome measures: gestational length and low birth weight. While the short gestational length of the mouse is convenient in the terms of the length of experiments, it poses some difficulty to precisely distinguish premature from full term (see Materials and Methods). Mice were chosen as the model for adverse perinatal outcomes subsequent to maternal UTI for the following reasons: 1) the major histocompatibility complex classes are known such that outbred strains can be chosen to invoke fetal tolerance as occurs in humans, 2) short gestation period (20-21 days), 3) embryonic stages are well classified, 4) long-standing model for human UTIs.

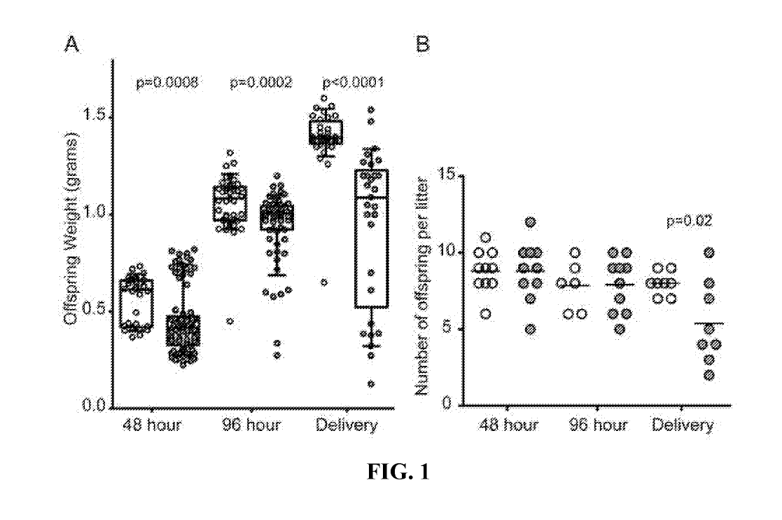

While prior experiments have detailed septic murine models producing adverse fetal outcomes, the inventors report herein the first model utilizing a local infection (cystitis) to study perinatal detriment. Pregnant mice were given experimental UTI by introduction of approximately 10⁷ viable uropathogenic Escherichia coli (UPEC) transurethrally into the bladder or sham treated with phosphate buffered solution (PBS) at embryonic day 14 as previously described. Hung et al., Nat Protoc 4, 1230 (2009). The weight of each of the offspring was measured on the day of harvest (FIG. 1). Sham treated mice fostered more robust fetal growth than the mothers that received experimental UTI (FIG. 1). At 48 hours post-infection, the median fetal weight from mothers that experienced sham infection was 0.617 g (±0.122), while the median fetal weight from mothers with UTI was significantly lower (0.417 g±0.165) (p=0.0015). Fetal weight gain was observed over the next 48 hours in most mothers, but remained stunted 96 h following introduction of maternal experimental UTI (1.004 g vs 1.083 g) (p=0.0014). The greatest disparity in fetal weight was observed at term delivery. At parturition, the median pup weight from infected mothers was 1.090 g (±0.404), which was significantly lower than the median pup weight of 1.395 g (±0.157) from mothers who were given only sham treatment (p=<0.0001). All mothers delivered at or later than embryonic day 19, making them full term deliveries. Therefore, the changes in weight cannot be attributed to differences in gestational length. Thus, the experimental evidence strongly suggests that adverse perinatal outcomes (i.e., low birth weight offspring) are the direct result of maternal UTI.

Cystitis Remains Localized During Pregnancy.

In most clinical cases, the causative agents of UTIs during pregnancy remain within the urinary tract. The genitourinary tissues, fetus and blood from pregnant mice were tested for presence of viable UPEC to determine the tissue distribution of UPEC following introduction of UTI. No bacteria were detected in any tissues from the sham-treated mice. A robust bacterial burden was observed in the bladder of mothers that received UPEC throughout pregnancy and parturition (Table 1). The levels of the infection were sustained longer than in most non-pregnant mice and were reminiscent of a chronic infection observed in non-pregnant mice. Hannan et al., PLoS Pathog 6, (2010). Viable bacteria were not detected in the majority of the kidneys from mice that were given experimental cystitis. One mouse at each time point demonstrated bacterial burden in the kidney that was just above the level of detection (10³ colony forming units/kidney pair), indicating that the urinary tract infection was restricted to the lower tract (bladder) throughout pregnancy. To ensure that the UPEC remained localized within genitourinary tissues and did not systemically spread, the viable bacterial burden within the uroplacental tissue, blood, and fetal tissues were enumerated. No viable bacteria were detected (Table 1), indicating that UPEC did not disseminate systemically and that other bacteria did not gain access to the reproductive organs. Thus, the fetal detriment detailed above is likely not due to direct bacterial invasion of the uteroplacental tissue, but rather an inflammatory response disseminated to the uterus via cellular and/or soluble immune effectors.

TABLE 1
Bacterial burden of the bladder, kidney, and uterus of infected and
sham-treated mothers at 48 h, 96 h, and term are reported as colony
forming units per organ with the standard deviation.

| Time | Bladder | Kidney | Uteroplacental | Blood |
|---|---|---|---|---|
| 48 hour | 6 × 10⁵ ± 670 | N.D. | N.D. | N.D. |
| 96 hour | 4 × 10⁴ ± 3887 | N.D. | N.D. | N.D. |
| Parturition | 1 × 10⁴ ± 22 | N.D. | N.D. | N.D. |

N.D = not detected (Limit of detection is 10³ cfu)

Cellular Inflammatory Influx in the Bladder During UTI.

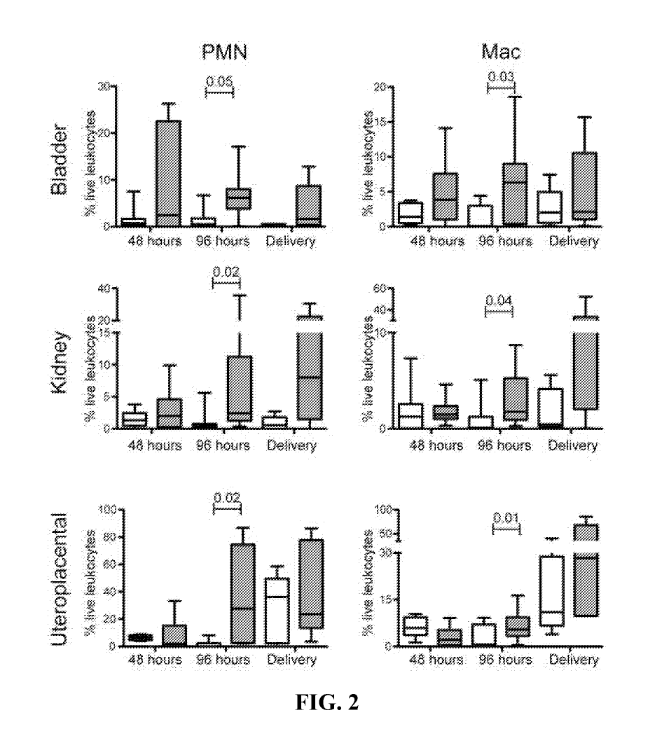

Although the infiltration of both PMNs and macrophages to the bladder during cystitis has been described, the extent of phagocytic recruitment to the bladder of a pregnant mouse is not well characterized. The inventors next evaluated the magnitude and type of proinflammatory cells, which were recruited to the bladder during experimental UTI in pregnant mice. Initially the bacterial burden, myeloid infiltrate and lymphoid infiltrate in each of the genitourinary tissues was examined. While bacteria (Table 1) and myeloid cells (FIG. 2) were enumerated, the inventors did not detect a significant magnitude of lymphoid cell recruitment within the portions of the tissue examined at selected time points (data not shown). At 48 hours post-infection, both PMNs (Gr-1^{+hi}, CD11b⁺, Ly6C^{+hi}) and macrophages (Gr-1^{+low}, CD11b⁺, Ly6C^{+lo}) were elevated in the bladder of infected pregnant mice compared to sham pregnant mice. At 96 hours post-infection, the number of phagocytic cells increased significantly [PMNs (p=0.023) and macrophages (p=0.012)] in the bladder. As the mothers went to term, the levels of both PMNs and macrophages remained elevated for those infected, but declined as the infection was resolved (Table 1). The sustained inflammatory responses during pregnancy are consistent with the sustained bacterial burden for the bladder described above. The magnitude of PMN infiltrate is higher in the bladder of pregnant mice (p=0.01) than in non-pregnant mice, further suggesting that pregnancy produces a distinct inflammatory environment in response to an extrauterine infection (i.e., UTI). Horvath Jr. et al., Microbes Infect, 13(5):426-37 (2011).

Cellular Inflammatory Influx in Other Genitourinary Tissues During UTI.

The inventors next evaluated whether the induction of localized cystitis in the bladder would elicit a cellular immune response in adjacent genitourinary tissues. Renal tissue demonstrates a similar cellular pro-inflammatory infiltrate as observed in the bladder (FIG. 2), despite the lack of viable UPEC in the organ (Table 1). The magnitude of both PMNs and macrophages increased over time with the 96 h time point revealing the most significant differences (p=0.006 and p=0.004, respectively). With respect to the uterus, a sustained elevation of PMNs and macrophages in the uteroplacental was observed at all time points analyzed, (FIG. 2). At 96 hours post-infection the number of phagocytes was significantly greater in the infected group (for PMNs, p=0.012 and for macrophages, p=0.016). However, by the time of natural delivery, the difference in PMN infiltration into the uterus lessened (p=0.56). In contrast, there was a considerable rise in macrophage presence in the infected group (note change in scale of y axis). However, this massive influx of macrophages was not significantly different to the recruitment of macrophages in the non-infected mothers. An increased of phagocytic influx into uterus of non-infected has been previously demonstrated. Sukhikh et al., Bull. Exp. Biol. Med. 134, 107 (August, 2002). Furthermore, the study suggested a functional role of phagocytes during and after the natural course of parturition. Interestingly, the inventors experiments indicate that inflammation-associated cellular effectors are present in tissue that lack UPEC colonization, (i.e., uteroplacental tissue with no viable UPEC) suggesting that either soluble bacterial factors or host-related transient modulators result in such an inflammatory environment.

Mature Dendritic Cells (mDC) Increase During UTI.

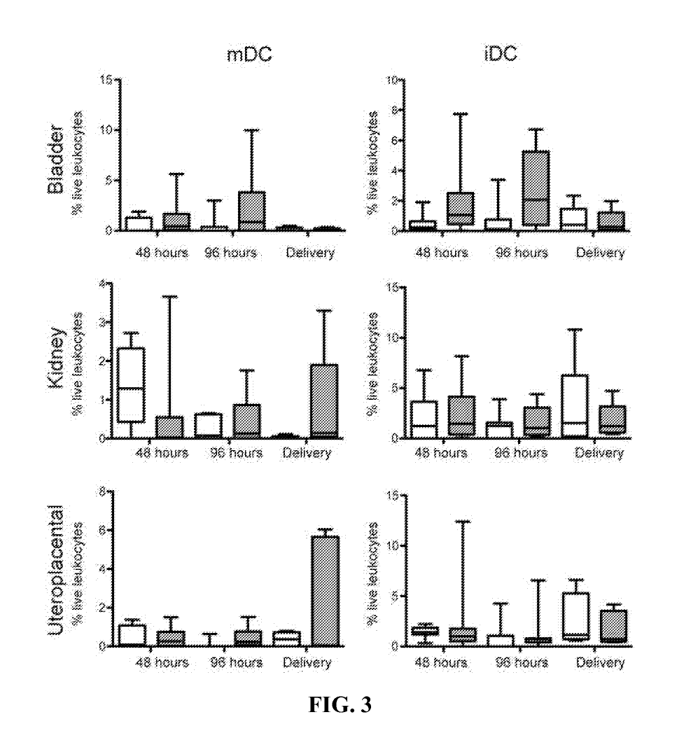

The maturation state of DCs dictates the success or failure of outbred pregnancies [42]. In this study, a statistically significant increase in the magnitude of mDCs (CD11c+, MHC II+med-hi) was detected in the bladders of infected mice 48 and 96 hours post-infection (gestational day 16 and 18, respectively) (FIG. 3). The presence of mDCs in the uterus indicates that inflammatory signals have expanded beyond the bladder and initiation of adaptive immunity has occurred (i.e., uterine mDCs). Interestingly, the percentage of iDCs, (CD11c+, MHC II+lo) which are important for maintenance of fetal tolerance, remained unchanged in uteroplacental tissues at all of the post-infection time points analyzed (FIG. 3). The similar levels of iDCs suggest that, at least, portions of fetal tolerance are intact in the presence of maternal UTI. In addition, the data suggest that mDCs may have matured elsewhere (e.g., bladder) and may have trafficked to the uterus upon infection of the urinary.

Serum Cytokines are Elevated During UTI.

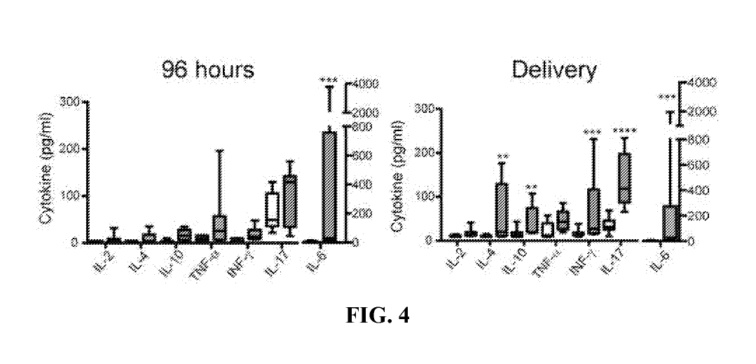

As serum levels of pro-inflammatory cytokines are elevated during UTI (Sheu et al., Cytokine 36: 276-282 (2006)), the inventors were interested to determine whether specific cytokines are associated with maternal UTI-mediated IUGR-LBW. Serum was collected from sham-treated and experimental UTI cohorts at the time of tissue harvest for evaluation of pro-inflammatory cytokine levels as described in Materials and Methods. While cytokines were elevated in the experimental UTI cohort as compared with the sham cohort at 48 hours, none of the individual comparisons reached statistical significance (data not shown). Circulatory pro-inflammatory cytokines were greater in mothers experiencing experimental UTI than in those mothers in the sham cohort at 96 hours post-introduction of UTI and at parturition (FIG. 4). At 96 hours post introduction of infection, only the values for IL-6 were significantly greater in mothers that received experimental UTI compared to mothers in the sham cohort (p=0.0028) (FIG. 4). When the magnitude of serum IL-6 was correlated to the weight of each individual fetus at 96 h post infection, the highest levels of IL-6 were observed in those infected mothers with the lowest weight offspring. At delivery, the magnitude of IL-6 remained significantly elevated (p=0.001) as well as the magnitude of IL-4 (p=0.04), IL-10 (p=0.03), INF-γ (p=0.008) and IL-17 (p=0.0003) (FIG. 4). These data demonstrate that a robust systemic immunological response, as measured by the magnitude of serum cytokines, occurs downstream of maternal UTI at a time when the bacteria have transitioned from the acute to the latent infection.

Transcriptional Changes in Placenta Resulting from Maternal UTI.

In order to identify early changes in the placenta that lead to fetal demise, the inventors compared transcriptional changes in the presence and absence of experimental UTI. Placenta and uterus were harvested on gestational day 16 (48 hours post UTI) from mice from the sham cohort and mice from that received experimental UTI on gestational day 14. The tissue samples were processed for extraction of mRNA and subjected to Agilent microarray analysis (Methods and Materials). There were no significant differences in the transcriptional profiles between the uterus tissue in the presence or absence of infection (data not shown). Within the placental tissues, there were 257 transcripts that ranged in differential expression values ranging from 4.7-810 fold (Table 2) (full raw data deposited into the MIAME compliant GEO database, accession GSE32028). Cathepesin Q, as well as a number of other apoptotic factors, demonstrated robust increase in expression (up to 810 fold) in the presence of maternal UTI. Placental levels of prolactin were also significantly increased in the presence of maternal UTI. There was an increase in the expression of the leptin receptor in the placenta of mothers that receive experimental UTIs. None of the genes represented in the transcriptional analysis were directly related to activation or recruitment of the innate immune response (e.g. TRL receptor, cytokine production, chemokine production), which corroborates the observation that the reproductive organ is sterile. Furthermore, the lack of primary inflammatory mediators suggests that bacterial antigens do not appear to escape the bladder to gain access to the reproductive organ to initiate the proinflammatory responses observed in the uteroplacental unit.

TABLE 2
The top 4 families that demonstrate increased transcription
in each individual placenta at 48 hours following introduction
of maternal UTI are indicated, along with the number of times
each family was represented and the fold change in expression.

| | | Number of | Fold | | |
|---|---|---|---|---|---|
| Gene Name | Function | Occurrences | Increase | FDR | AdjP |
| Cathepsin Q | Apoptosis | 1 | 810 | 34.97 | 0.06641 |
| | from ROS | | | | |
| Prolactin | Maintain fetal | 22 | 28-784 | 34.07- | 0.06641- |
| Family | tolerance | | | 35.01 | 0.08757 |
| Leptin | Fetal weight | 9 | 71-93 | 34.65- | 0.06641 |
| Receptor | gain | | | 34.97 | |
| Cathepsins | Apoptosis | 1 each | 81-286 | 34.97- | 0.06641- |
| (3, 6, M, J, R) | | | | 35-01 | 0.07409 |
| Ceacam | Cell adhesion | 5 | 56-568 | 34.65- | 0.06641- |
| | molecules | | | 35.01 | 0.07409 |

### Discussion

The inventors have demonstrated that a non-disseminated UTI is associated with adverse perinatal outcomes. Kaul et al. presented a mouse model of pyelonephritis-induced preterm birth and low birth weights while investigating the virulence of the Dr adhesion. Kaul et al., Infect Immun 67: 5958-5966. In contrast to the inventors' model, the mothers became septic and all of the fetuses became infected, presumably due to the use of a pyelonephritic UPEC strain (compared to the inventors' cystitis strain) and the use of immunocompromised TLR4-deficient mothers. Due to the dissemination to the reproductive organ, the model presented by Kaul et al. provides additional insight into the effects of intrauterine infection on fetal development. In our system, the offspring of mothers that experienced experimental UTI displayed up to 80% decrease in fetal weight when compared to non-infected mothers. This phenomenon occurs as early as 48 hours post introduction of cystitis and continues throughout gestation. An important feature of the inventors' model of adverse perinatal outcome model is that localized cystitis induced delayed infiltration of PMNs and macrophages in distal organs. The inventors demonstrated that bladders from mice with experimental UTI had an expected increase of professional phagocytes as a result of localized infection; however, inflammatory cells were also observed in the kidney, even in the absence of viable bacteria. Furthermore, a robust cellular inflammatory response was observed in the uteroplacental tissue of those mice with experimental UTI when no viable bacteria were recovered (the same tissue sample tested for both bacteria and cellular infiltrate). In fact, there was a correlation between PMN infiltration and diminished fetal weight gain, suggesting that the presence of the PMNs may contribute to the IUGR-LBW observed in the presence of non-disseminated UTI. The cellular inflammatory response in the pregnant reproductive organ was more severe than the kidney and in the naïve uterus, suggesting that the uteroplacental tissue is no longer privileged and may be more susceptible than other adjacent genitourinary organs to systemic changes in immune status.

The evidence indicates that the placenta is progressing through apoptotic death as a consequence of a strong cellular influx of immune cells into the reproductive organ. Cathepsin Q is a placental specific apoptotic factor that induces necrotic cell death in the presence of reactive oxygen species-mediated DNA damage such as produced by PMNs and macrophages. The inventors hypothesize that the induction of Cathepsin Q in the UTI cohort indicates that the influx of professional phagocytes is inducing DNA damage within the placental cells. High serum prolactin is associated with miscarriage in humans (Hirahara et al., Fertility and sterility 70: 246-252 (1998)) and prolactin is involved in the T-cell functions associated with maintenance of fetal tolerance. Handwerger et al., Trends in endocrinology and metabolism: TEM 3: 91-95 (1992). Whether prolactin levels are elevated as a consequence of the phagocyte infiltration as a means to protect the fetus from the maternal immune response or whether the levels disrupt the T-cell functions that lead to inflammatory cell infiltrate are under investigation. Leptins are important in proper fetal weight gain. The increased production of the leptin receptor suggests that the placental unit is attempting to acquire more nutrition for the developing fetus. These observations provide insight into the molecular mechanisms that underlie intrauterine growth restriction as a result of maternal UTI.

Cytokines measured in the serum of our infected pregnant females demonstrated the absence of Th2 effectors characteristic for healthy pregnancy, and thus, both Th1 cytokines (INF-γ, TNF-α) and Th17 cytokines (IL-17) are significantly elevated at delivery. Furthermore, IL-6 and TGF-β are key factors in the development of Th17 cells. Similar systemic inflammation along with an imbalance of Th1 and Th2 cells in the uterus is a dominant component implicated in the pathogenesis of pre-eclampsia. Toldi et al., American journal of reproductive immunology, 66(3): 223-9 (2011). This change from Th2 to Th1 should alter DC function and compromise fetal tolerance. However, the inventors did not detect significant changes in uteroplacental iDCs, thus, the inventors favor the hypothesis that mDCs may traffic to adjacent organs via hematogenous route. Multiple mechanisms by which DCs can mobilize to lymph node or spleen, return to the blood and enter other organs were reviewed by Randolph et al. Randolph et al., Curr Opin Lipidol 19: 462-468 (2008). Reverse trafficking might facilitate the spread of antigens from tissue to tissue carried by DCs serving as Trojan horses. However, the inventors transcriptional analysis of the placenta indicates that the placenta is not the primary site of immune activation under these experimental conditions. It is also possible that cellular innate responses result in increased levels of cytokines such as TNF-α that could stimulate DC maturation in distinct and distant urogenital tissues, which would in turn, promote Th1/Th17 responses and manifest in a more robust systemic inflammation. Indeed, the inventors were able to detect an increase in mDC in the uteroplacental tissue, and also elevated inflammatory cytokines in the serum, which might impact the type and severity of the inflammatory responses. The implication of a remote infection producing sufficient inflammatory stimuli to restrict adequate fetal development has numerous clinical implications. Moreover, these observations suggest that local infections result in the inflammation in a number of non-infected organs, which may also impact the function of those organs. In this particular case, the systemic inflammation has a dramatic effect on an organ function (i.e. the uteroplacental unit) leading to intrauterine growth restriction and low birth weight.

In conclusion, the inventors murine model of maternal non-disseminated UTI-induced adverse perinatal outcomes provides a platform to further elucidate the inter-organ cross talk that occurs during localized infections.

### Materials & Methods

Mice and Optimized Mating for Generation of Outbred Pregnancies.

4-6 week old C57Bl/6 female mice were obtained from Jackson Lab, (Bar Harbor, Me.). Females were outbred with C3H/HeN males less than 1 year of age purchased from Harlan Labs (Indianapolis, Ind.). Forty-eight hours prior to the overnight mating sessions, males and females were exchanged in the cages to allow the mice to become familiar with their future mate's scent in an attempt to increase the rate of conception. To synchronize impregnation and maintain precise infection and delivery dates, 2 females were housed with 1 male (in the female's cage) overnight for less than 16 hours. Therefore, successful pregnancies were determined by maternal weight gain following cohousing. Female mice that did not become pregnant as determined by every other day weight measurements were returned to the mating rotation every 2-3 weeks until conception occurred. Females were deemed pregnant if they had gained at least 2 grams over a 1-week period. The distinct histocompatibility complex classes chosen will invoke fetal tolerance to prevent fetal rejection. The mating combination resulted in the typical number of fetuses with a gestational length of 20 days. With this mating pair combination, we observed a normal distribution of fetus number (8-10) and gestational length (typically 19-21 days). Taken together with an average rate of impregnation of 30%, these variables indicate that maternal fetal tolerance prevented rejection of the paternal allo-antigens resulting in a successful pregnancy. The presence of fetal tolerance in our outbred model is further supported by other investigations that demonstrate fetal rejection using a different outbred mating pair that does not invoke fetal tolerance. Maintenance of all mice was in strict accordance of the Institutional Animal Care and Use Committee (IACUC) rules and regulations. All animals are housed in accordance with USDA guidelines for the care and housing of laboratory animals, and USDA officials routinely inspect the facilities. Specifically, the mice had a normal 12-hour light-dark cycle and were maintained on standard chow diet (Harlan Laboratories). The mice were housed using ventilated cages, corncob bedding, and proper enrichment with changes on a two-week basis. As such, at most, the cages were changed once during the experimental period. The mice were also sequestered from being inadvertently disturbed by human interactions due to housing in a separate containment unit that was only opened when the pregnant mice were monitored. The experiments presented in this manuscript are approved (AR08-00039) by The Research Institute at Nationwide Children's Hospital Institutional Laboratory Animal Care and Use Committee (Welfare Assurance Number A3544-01).

Infection/Bacterial Strains.

UTI89/pANT4 (Justice et al. Proceedings of the National Academy of Sciences of the United States of America 101: 1333-1338 (2004), a prototypical UPEC strain obtained from a patient with cystitis which contains an episomal plasmid asserting ampicillin resistance, was used for all studies. Pregnant female mice were transurethrally inoculated with 50 μl of ˜108/mL UTI89/pANT4 or sterile phosphate buffered saline (PBS) as previously described. Hung et al., Nat Protoc 4: 1230-1243 (2009).

Tissue Processing.

Following isoflurane anesthesia per IACUC protocols, renal arteries of the pregnant mice were severed and blood was aspirated into tubes containing EDTA. Bladder, kidneys, and uteroplacental tissues were minced into small pieces with scissors and were digested for 25-30 minutes at 37° C. under slight agitation. Bladders and kidneys were digested in 0.5 mg/ml collagenase and 100 μg/ml DNase I in RPMI 1640 medium (Invitrogen, Grand Island, N.Y.) containing 0.5% heat-inactivated fetal calf serum (PAA Laboratories, Pasching, Austria) and 20 mM HEPES as previously described. Engel et al., Infect Immun 74, 6100 (November, 2006). The uteroplacental tissues were digested in a HBSS solution containing 1 mg/ml collagenase type IV, 0.2 mg/ml DNase 1,200 U/ml hyaluronidase, and 1 mg/ml bovine serum albumin/fraction V as previously described. Blois et al., Biol Reprod 70, 1018 (April, 2004). After enzymatic dissociation of tissues, an aliquot of each organ (bladder, kidney, or uterus) or undiluted blood was serially diluted in PBS and plated on LB agar and LB agar containing ampicillin to enumerate bacterial burden. The number of colonies was indistinguishable on LB agar and LB agar containing ampicillin.

Analysis of Cellular Inflammation.

Cell suspensions were washed in FACS buffer and filtered through a 70 μm nylon filter (Becton Dickinson, Franklin Lakes, N.J.) to remove cellular debris. One mL of ACK lysing buffer (Invitrogen, Grand Island, N.Y.) was added to the kidney, spleen, and bone marrow to lyse red blood cells. A discontinuous 20, 40, and 80% Percoll gradient was prepared as described (Nagaeva et al., Am J Reprod Immunol 47: 203-212 (2002)) according to manufacturers recommendations (GE Healthcare Biosciences, Pittsburgh, Pa.). One ml of single cell suspensions of kidneys and uterus were underlayed in 80% Percoll layer and centrifuged at 500×g for 25 min at room temperature. The band between the 40 and 80% layer was collected and washed twice in PBS. Prior to antibody staining, single cell suspensions of each sample were incubated with Fc blocking antibody (eBiosciences, San Diego, Calif.), to minimize non-specific antibody staining. The following antibodies were used to discern specific cellular populations: anti-CD11b-phycoerythrin (M1/70), anti-Gr-1-phycoerythrin-cy-5.5 (RB6-8C5), anti-Ly6C-Alexaflour 488 (ER-MP20), anti-CD11c-allophycocyanin (N418), anti-major histocompatibility complex class II-Alexa 700 (M5/114.15.2), anti-major histocompatibility complex class II-Biotin (KH74), anti-allophycocyanin-cy-7 streptavidin, anti-F4-80-phycoerythrin-cy-7 (BM8) (all from eBiosciences, San Diego, Calif. except anti-MHC II-Biotin, BD Pharmingen, San Diego, Calif.). Single color controls for each antibody were used with either bone marrow or spleen for fluorescence compensation. A viability discrimination marker was used according to the manufacturers' instructions (Violet Live/Dead kit, Invitrogen, Carlsbad, Calif.) to exclude dead cells from subsequent analysis. Cellular data were collected with a BD LSR II flow cytometer (Becton Dickinson, San Jose, Calif.) and analyzed with Flowjo software (Tree Star, Ashland, Oreg.). Statistical analysis and graph constructions were made using Prism 5.0 (GraphPad Software, Inc, La Jolla, Calif.).

Bacterial Cultures.

Each organ sample, including blood, was plated on LB plates containing ampicillin sulfate (125 μg/ml, Fischer Scientific, Fairlawn, N.J.). Serial dilutions of each organ were plated in consecutive rows on LB agar and LB agar containing ampicillin to enumerate bacterial burden. Undiluted blood samples were also plated. Colony counts were then recorded for any growth and labeled as none detected if no growth was witnessed. The number of colonies was indistinguishable on LB agar and LB agar containing ampicillin.

Cytokine Analysis.

Maternal serum was analyzed for various cytokine levels using a Th1/Th2 and a Mouse Inflammation Cytokines Bead Arrays (both from BD Biosciences, San Jose, Calif.). Samples taken at the time of harvest from severed renal vessels were spun for 10 minutes at 10,000 rpm. Serum was then collected and frozen at −80° C. for batch testing. Fifty microliters of serum was used for each sample tested. If more than 50 μl was available, samples were run in duplicate. An LSR II cytometer was then used to measure the following: IL-2, IL-4, IL-5, IL-6, IL-10, IL-12, IL-17, IFN-γ, and TNF-α. Software included in the kits was used for conversion into pg/ml.

mRNA Extraction.

Samples of the placenta and the uterus were taken on gestational day 16 from 4 mice. Two mice received experimental UTI or shame inoculation on gestational day 14. Tissue samples were homogenized in 1 ml TRIZOL® Reagent per 50-100 mg of tissue using power homogenizer Polytron. The homogenized samples were incubated for 5 minutes at room temperature and then 0.2 ml of chloroform per 1 ml of Trizol were added to the tube. Total RNA was isolated from the homogenized samples with TRIZOL reagent according to the manufacturer's recommendations.

Microarray Analysis.

Two-color gene expression analysis was performed in-house by the Biomedical Genomics Core. In outline, 500 ng of total RNA was amplified and labeled with either Cy3 (uninfected control samples, n=2) or Cy5 (infected test sample, n=2) using the Low Linear Amplification Kit (Agilent Technologies, CA). This labeling reaction produced 7-10.0 μg of Cy3-labeled cRNA (anti-sense), by first converting mRNA primed with an oligo (d)T-T7 primer into dsDNA with MMLV-RT and then amplifying the sample using T7 RNA Polymerase in the presence of Cy3-CTP. After purification, 825 ng of each the test and control cRNA was fragmented and co-hybridized to the Whole Mouse Genome Oligo Microarray (AMADID 04868; Agilent Technologies, CA) array for 17 hr. at 65° C. This array consists of 44,000 60-mer oligonucleotides, representing 21,609 known genes represented by 33,661 transcripts.

Microarray slides were washed and then scanned with an Agilent G2505C Microarray Scanner. Images were analyzed with Feature Extraction 10.7 (Agilent Technologies, CA) in two color gene expression mode. Median foreground intensities were obtained for each spot and imported into the mathematical software package “R”. The intensities were corrected for the scanner offset but not further background corrected. The dataset was filtered to remove positive control elements. Using the negative controls on the arrays, the background threshold was determined and all values less than this value were set to the threshold value. Finally, the data were global loess normalized using the LIMMA microarray processing package in “R”. Fold change values were then calculated for each element on the array and averaged for the replicate samples. Genes with a fold change >2 fold up or down were considered differentially expressed. All microarray data is MIAME compliant and the raw transcriptional data has been deposited into the MIAME compliant database, GEO, accession number GSE32028.

Statistical Analysis:

Exact values (symbols) or median values (graphs) are represented in each figure. Statistical significance was determined using two-tailed non-parametric Mann-Whitney U test (GraphPad Software, Inc, La Jolla, Calif.).

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method for assessing a risk of experiencing an adverse perinatal outcome in a subject comprising:
(a) determining a level of at least one cytokine in a biological sample from the subject by analyzing the biological sample using a cytokine analytic method; and
(b) comparing the determined level of the at least one cytokine in the subject's biological sample with at least one predetermined value,
wherein a subject whose level of at least one cytokine is greater than the predetermined value is at increased risk of experiencing an adverse perinatal outcome.

2. The method of claim 1, further comprising providing a report indicating the subject is in need of therapy to decrease the risk of an adverse perinatal outcome if levels of at the least one cytokine are higher than at least one of the corresponding predetermined values.

3. The method of claim 1, wherein the at least one cytokine is a pro-inflammatory cytokine.

4. The method of claim 3, wherein the pro-inflammatory cytokine is selected from the group consisting of interleukin-6, interleukin-17, and tumor necrosis factor alpha (TNF-α).

5. The method of claim 1, wherein the at least one cytokine is a circulatory cytokine, urinary cytokine, or both.

6. The method of claim 1, wherein the biological sample is selected from the group consisting of blood, serum, and urine.

7. The method of claim 1, wherein the biological sample is urine.

8. The method of claim 1, wherein the subject has been diagnosed with a urinary tract infection.

9. The method of claim 1, wherein the population of control subjects is pregnant females.

10. The method of claim 9, wherein the pregnant females are apparently healthy pregnant females.

11. The method of claim 10, wherein the apparently healthy pregnant females have been diagnosed as not having an infection.

12. The method of claim 11, wherein the infection is a urinary tract infection.

13. The method of claim 1, wherein the predetermined value is a single normalized value or a range of normalized values and is based on the at least one cytokine level in comparable biological samples from the control subjects.

14. The method of claim 1, wherein the predetermined value is a single representative value or a range of representative values and is based on the at least one cytokine level in comparable biological samples from the control subjects.

15. The method of claim 1, further comprising administering an effective amount of a therapeutic agent comprising an anti-inflammatory agent or a progestational agent to a subject identified as having an increased risk of experiencing an adverse perinatal event.

16. The method of claim 15, wherein the therapeutic agent is selected from the group consisting of cathepsin Q, prolactin, leptin receptor, cathepsin 3, cathepsin 6, cathepsin M, cathepsin J, cathepsin R, ceacam, and anti-inflammatory agents.

17. The method of claim 1, wherein the cytokine analytic method is an immunoassay.

18. A method for evaluating therapy in a subject suspected of being or diagnosed as being at increased risk for an adverse perinatal outcome, comprising:
(a) determining a level of at least one cytokine in a biological sample obtained from the subject prior to therapy to decrease the risk of an adverse perinatal outcome and during or after therapy using a cytokine analytic method, and
(b) comparing the level of at least one cytokine in a biological sample obtained from the subject prior to therapy to the level of at least one cytokine in a biological sample obtained from the subject during or after therapy,
wherein a decrease in the at least one cytokine level in the subject's biological sample taken after or during therapy as compared to the at least one cytokine level in the subject's biological sample taken before therapy is indicative of a positive effect of the therapy.

19. The method of claim 18, wherein the therapy comprises administering an effective amount of an anti-inflammatory agent or a progestational agent.

20. A kit comprising: one or more reagents for determining a level of at least one cytokine in a subject; and information for assessing the subject's risk of having an adverse perinatal outcome.
